(19) United States
(12) Patent Application Publication
(10) Pub. No.: US 2008/0124731 A1
(43) Pub. Date: May 29, 2008

(54) Natural Food Tracer

(76) Inventors: Marco Pancaldi, Spilamberto MO (IT); Luca Fontanesi, Crevalcore BO (IT); Alessandro Salvi, Borgio Verezzi SV (IT); Vincenzo Russo, Reggio Emilia RE (IT); Roberta Davoli, Reggio Emilia RE (IT); Elena Carboni, Zola Predosa BO (IT)

Correspondence Address: NIXON & VANDERHYE, PC, 901 NORTH GLEBE ROAD, 11TH FLOOR, ARLINGTON, VA, 22203, US

(21) Appl. No.: 11/920,261

(22) PCT Filed: May 15, 2006
(86) PCT No.: PCT/IB2006/051516

§ 371 Date: Dec. 4, 2007

(30) Foreign Application Priority Data
May 13, 2005 (IT) RM2005A000235

(51) Int. Cl. C12Q 1/68; C12P 19/34

(52) U.S. Cl. 435/6; 435/91.1

(57) ABSTRACT

The present invention relates to tracers for foods comprising natural nucleotide sequences, methods for the production thereof, uses of said tracers and food products labeled with said tracers.

The present invention relates tracers for foods comprising natural nucleotide sequences, methods for the production thereof, uses of said tracers and food products labelled with said tracers.

### STATE OF THE ART

The problem of recognizing the origin of products, of the fight against counterfeits and of the possibility of tracing the exact origins of a product is a current problem and several methods and tracing systems have been created for its solution.

As an example, the international application WO 03/087765, discloses a method for tracing animals and food products deriving from the same, by a fingerprinting of the individual DNA. The aim of the disclosed method is to permit the traceability of the food product through all the food chain back to the origin system in the producing farm. The method disclosed in WO 03/087765 provides a genetic profile of a parental animal or of a product of a parental animal for the traceability, comprising obtaining a sample of said parental animal or of said product of the parental animal, genotyping said parental animal or said product of the parental animal using a system based on DNA markers in order to obtain a profile of the genomic sequence of said parental animal or of said product of the parental animal from the system based on the use of DNA genetic markers. Said profile would allow the determination of the system or of the farm of origin of said parental animal or of said product of the parental animal. The genotyping may be realizable through microsatellites, SNPs, AFLPs, deletions, insertions and requires the genotyping of the animal to be traced. This can be done, by way of example, by using polymorphisms of the same animal, therefore establishing means for obtaining the labelling of the product. Obviously, the method described can be in part applied to meat coming from farms because the genotyping of a single breeding male (bull, pig, etc.) provides information for half of the genetic inheritance of hundreds of animals born from said parental animal. However, said tracing method is hardly applicable to food products such as dairy or vegetable products. Moreover, also in the case of the parental animals, the death of the animals, and the absence of useful seminal material and/or egg cells, would inevitably bring to the necessity of genotyping new animals. Consequently, the method for the identification of animals and/or of their products requires subsequent adjustments and further analysis and may not be used in a system wherein the genotype of the parental animals is unknown.

US application US2004/0137458, describes a tag made of single strand nucleic acids and the use of said tags for monitoring, detect, or trace substances comprising said tag. The said patent application discloses the fact that the described tags may be used to label food products, however, said application does not indicate how the invention should be carried out in order to label food products.

International patent application WO 00/44891, discloses an ink comprising DNA fragments amplified with primers comprising 3′ and 5′ labelling groups in order do identify products labelled or coloured with said ink.

All the above mentioned examples disclose labelling means comprising sequences that are either artificial and/or labelled with substances that are non suitable for food, or comprising sequences that require each time the genotyping of a subject at the beginning of the food production chain to be traced in order to be defined.

Up to date, it does not seem that the state of the art provides for tracers suitable for agricultural and food products, that are reliable, rapidly detectable, and that do not depend from the animal or from the plant/s directly used in the production of the food product as in the case of the genotyping of the animal to be traced described above.

### SUMMARY OF THE INVENTION

The present invention provides a tracer for foods of natural origin that does not require a fingerprinting of the product itself, said tracer being suitable also for agro-food products such as, e.g., typical products, e.g. Registered Designation of Origin (R.D.O.), Protected Designation of Origin (P.D.O.), Protected Geographical Indication (P.G.I.), Guaranteed and Registered Designation of Origin (G.R.D.O.) products.

The marker of the invention is based on the use of DNA polymorphic sequences, such as, e.g., SNPs (Single Nucleotide Polymorphisms) or microsatellites such as SSRs (Single Sequence Repeats) or STRs (Single Tandem Repeats) etc., of plants or animals, or bacteria, or fungi, or yeasts, or other micro-organisms normally used for edible products, as markers. Said markers being comprised in a tracer for foods and hence being suitable also for agro-food products as they do not contain artificial sequences or marker molecules that are not suitable to be used in edible products or not desirable in high quality typical products such as, e.g., R.D.O., P.D.O., P.G.I., G.R.D.O. products.

Hence, object of the invention are:

a tracer for foods comprising DNA sequences of natural origin and/or natural material comprising said sequences and suitable excipients, characterised in that:

the said DNA sequences comprise one or more polymorphic sequences detectable by DNA amplification with one or more primers having an annealing temperature higher or equal to 50° C., said sequences comprising SNPs and/or microsatellites wherein said microsatellites have at least five alleles at each microsatellite locus, wherein the diversity index between the alleles of said loci must be higher or equal to 0.5 taking into account the number of alleles for each microsatellite and the relative frequency of each allele according to the formula:

DI=1−Σpᵢ²

where DI is the diversity index and pᵢ is the frequency if the i^{th} allele;

a method for the preparation of said tracer comprising the steps of:

a. selecting a plant variety and/or a breed or animal line or a line, a strain or a species of fungi, bacteria, yeast or other micro-organisms;

b. extracting the DNA of one or more specimen of the same variety, line, breed, strain or species selected at a., performing an homogeneity test for each variety, line, breed, strain or species and selecting the varieties, lines, breeds, strains or species showing homogeneity;

c. selecting one or more polymorphism for said varieties, lines, breeds, strains and/or species according to the following parameters:

Said polymorphisms must comprise SNPs and/or microsatellites and said microsatellites must have at least 5 alleles at each microsatellite locus

the diversity index between said loci must be higher than 0.5 taking into account the number of alleles at each locus and the relative frequency of each allele according to the following formula:

DI=1−Σpᵢ²

where pᵢ is the frequency of the i^{th} allele;

d. choosing one or more SNP and/or microsatellites selected at c. requiring annealing temperatures higher or equal to 50° C. for their DNA amplification;

e. isolating materials comprising the DNA of the variety/ies, line/s, breed/s, or species comprising the SNPs and/or the microsatellites selected in c. and d. and mixing said materials with suitable excipients;

the use of polymorphic natural sequences comprising SNPs and/or microsatellites selected according to the method described above for the identification and traceability of the origin of food products through all the food production chain in which the polymorphic sequences selected and comprised in the tracer of the invention are amplified by using specific primers;

a method for the detection of the food tracer of the invention comprising the steps of:

a. performing the microsatellite analyses with specific primers directly on the tracer or on DNA extracted from the said tracer, by one or more of the techniques selected from the group of: Polymerase Chain Reaction (PCR), Real Time Polymerase Chain Reaction (RT-PCR), Isothermal and Chimeric prime-initiated Amplification of Nucleic Acids (ICAN), Rolling Circle Amplification (RCA), Invader Technology, Single Primer Isothermal Amplification Technology (SPIA Technology), Loop Mediated Isothermal Amplification (LAMP), Ramification Amplification (RAM), Pressure Cycling Technology (PCT);

b. confronting the alleles obtained from the analyzed tracer with the alleles of a control sample of the said tracer;

foodstuff labelled with the tracer of the invention.

### DETAILED DESCRIPTION OF THE INVENTION

As indicated above, the tracer of the invention, comprises natural DNA polymorphic sequences. For the carrying out of the invention, said polymorphic sequences are comprised in the genomic DNA of any natural organism such as an animal, a plant, a bacteria, a fungus, a yeast or another micro-organism and shall meet the parameters indicated in the preceding section. Said DNA shall not necessarily be extracted from said organisms but may be used as contained therein, or contained in material deriving from said organisms. When the organism of choice is not comprised in the food product itself, the tracer can be applied externally to the product so not to adulterate in any way the organoleptic properties of the food product. If the organism of choice or derivatives thereof are contained in the product to be traced (by way of example olive tree-oil, vine-wine) the tracer may be directly inserted in the product in minimal quantities. As an example, a specific quality of olive tree or vine may be selected, and oil or wine produced therefrom may be introduced in the oil or wine to be traced.

In a further embodiment of the invention, the material selected for the tracer could be normally used for the manufacture of the product (by way of example plant flours admixed with pork fat in the process for the greasing of ham, seeds or fragmented seeds in the procedure for coating products, pepper grains in the procedure for the manufacture of salami, etc.)

Hence, the tracer of the invention, can be externally applied to the product (i.e. on the identification tags) and can also be applied to the product itself, or even admixed with edible inks, or integrated in the product during the procedure of the preparation of the said product.

For the aim of the invention, the selected polymorphisms must be detectable by one or more of the following DNA amplification techniques, such as: Polymerase Chain Reaction (PCR), Real Time Polymerase Chain Reaction (RT-PCR), Isothermal and Chimeric prime-initiated Amplification of Nucleic Acids (ICAN), Rolling Circle Amplification (RCA), Invaider Technology, Single Primer Isothermal Amplification Technology (SPIA Technology), Loop Mediated Isothermal Amplification (LAMP), Ramification Amplification (RAM) and Pressure Cycling Technology (PCT) and others. All the said techniques are well known in the art and all protocols for their carrying out are easily available to the skilled person. In particular, the microsatellites analysis (SSR) is at present considered a technique easily applicable for the tracing and the fingerprinting analysis, both on plants and on animals.

As indicated above, the DNA of the selected organism, must present polymorphic sequences detectable by DNA amplification and said sequences must have a high number of alleles (at least 5 for an optimal result) and said alleles must show a high heterozygosity. As an example, considering a polymorphism presenting 5 alleles, if one of said alleles is represented in the population with a too high frequency and the other four show a very low frequency, the locus will not be suitable to be selected for the tracer of the invention. The heterozygosity is, in the present invention, expressed as the diversity index. Said index must be higher than 0.5. Moreover, the polymorphic sequences of the tracer of the invention, must be amplifiable by the use of specific primers having an annealing temperature, in standard amplification conditions (MgCl₂ comprised between about 1 and about 3 mM) not inferior to 50° C.

In an embodiment of the invention said DNA sequences can be of plant, animal, bacterial, fungal, yeast or of other micro-organism origin.

In order to have DNA markers that are constant or stable for the selected biological materials, it will be advantageous, in the realisation of, the tracer of the invention, to use, when possible, sequences deriving from species showing agamic or autogamous propagation and showing a high inter-variety variability. A non limiting example of species presenting the above mentioned characteristic comprises olive, soft or hard wheat and rice.

The use of well studied species, for which a wide literature exists, will bring to a very fast selection of the polymorphisms suitable for the tracer of the invention.

In one embodiment of the invention, said microsatellite sequences are of a length comprised between 50 and 540 nucleotides or even comprised between 100 and 300 nucleotides. The advantage conferred by relatively short sequences (as the one indicated above), e.g. when the DNA is amplified by PCR, lies in a higher efficiency of the PCR on sequences of said dimensions with respect to longer sequences, and in an easier analysis of the above mentioned sequences.

The polymorphic sequences, amplified with anyone of the above mentioned techniques, can be easily analysed for their allelic composition with standard techniques known to the skilled person. In case of microsatellites, during the PCR, labelled primers that will allow a simple detection on a denaturing gel of the allelic composition of the amplified DNA, can be directly used. For the SNPs standard Real Time PCR can be used. In any case, the analysis of DNA polymorphisms is well known and available on laboratory handbooks and in a large amount of publications that are easily available to the skilled person. Any technique suitable for the analysis of said polymorphisms can be used to detect the tracer of the invention.

For the aim of the invention, the tracer may comprise more than one polymorphic sequences detectable by PCR techniques. It could hence be advantageous, in the carrying out of the invention, to select polymorphisms that may be amplified in a single multiplex PCR. If so, it will be necessary that the selected primers have similar annealing temperatures. In this embodiment of the invention, it will hence be advantageous that the selected polymorphisms can be amplified with primers having an optimal annealing temperature that does not differ for more than about 0.5 to 3° C. between primers for different microsatellites, i.e. a temperature that does not differ more than what indicated above, between the one for the primer having the lowest annealing temperature and the one for the primer having the highest annealing temperature. Furthermore, in the case of polymorphisms that may be amplified by multiplex PCR, it is advisable that the primers are highly specific for the sequence of interest. It will hence be advantageous to select primers having an annealing temperature higher or equal to about 60° C. Advantageously, one of the primers for each polymorphism may be appropriately labelled so to render the amplified sequence immediately detectable from the other amplified sequences, e.g. the different forward primers could be labelled, respectively, with Fam, Hex, Vic, Tet and other suitable markers known to the skilled person.

In another embodiment of the invention, different polymorphisms giving different sized products after amplification always comprised in the above mentioned ranges, could be selected. In this case, it will not be necessary to label the primers in a different way as the presence of a ladder indicating the length of the amplified sequences in a control gel, would allow the immediate identification of each amplification product.

The same concepts are obviously applicable to all the other DNA amplification systems according to the invention, allowing multiplex amplifications.

In order to obtain an effective tracing and tracking system, the DNA analysis, not only shall have a high selectivity, but shall also be feasible by analysing samples of various kinds (flours, liquids, seeds, extracts, freeze-dried products, and also what already indicated), that may constitute the biological tracer itself. The analysis must be possible after time has elapsed and after thermal and physical treatments to whom the products to be traced may be subjected.

The tracer of the invention may be manufactured in form of a solid, a solution, a powder, a flour, a lyophilised, a raw dry plant material, a liquid, depending on the food product to be labelled. The more suitable forms will be obvious to the skilled person. In the cases of an easy carrying out, the tracer can directly be in the form of seeds, oils, wines, flours and others. In other embodiments, materials deriving from the organism selected for the preparation of the tracer, and hence containing the DNA sequences according to the invention, may be admixed with other suitable food products, or edible excipients, or edible inks. The embodiments in which the tracer will particularly resistant in the storing processes (i.e. seeds, flours, oils etc.) will be particularly advantageous.

Obviously, the form of preparation that will render the tracer more easily applicable to the food product of choice, and in which the DNA comprised in the tracer will be more easily preserved, will be selected.

As an example, in the case of wheat, the tracer selected from time to time could be:
wheat flour added as such to the product,
wheat flour thawed in an edible ink and applied to the product,
seeds applied as such to the product;
freeze-dried wheat products applied to the product,
wheat panels applied to the product,
other products derived from wheat as such or processed in various ways.

In a particularly advantageous embodiment of the invention, the selected tracer will present the following characteristics:
practicality and easiness of application to the typical product;
easiness of preservation in time, both before the application to the typical product and during the preservation and the marketing of the same;
the DNA of the starting variety must remain inside the tracer, sufficiently integral notwithstanding the technological process suffered.

The same remarks are valid independently from the organism selected for the preparation of the tracer.

In the tracer of the invention, in order to avoid counterfeit, the name of the variety (or species, or line, or strain etc.) used for the preparation of the tracer as well as the corresponding DNA profiles, will be secret to the public. The silence on the variety used, renders the risk of counterfeit of the tracer of the invention impossible. In fact, in order to counterfeit the tracer of the invention, supposing that the publication of the organism selected for the production of the said tracer is mandatory (at least when the tracer is comprised in the food product), all the varieties of the species used in the preparation of the tracer of the invention should be tested in order to disclose the used one. Moreover, there would be the need to identify all the polymorphisms selected in order to use DNA presenting the same allelic composition of the tracer to imitate. When a plant species is used for the preparation of the tracer of the invention, as for a plant species there exist thousands of thousands of varieties spread in all the globe, it will be virtually impossible for the counterfeiter to reproduce the tracer of the invention.

In an embodiment of the invention, said tracer may contain material deriving from more than one variety of a same species as well as material belonging to different species of the same kingdom or even to different species of different kingdoms (animal, plant, fungal, protozoan).

A mixed set of polymorphisms could be hence selected for the detection of the tracer of the invention, e.g. a polymorphism for variety, species and kingdom so to render even less likely that the tracer of the invention is copied for counterfeit of the product on which the tracer is applied. Different lines or varieties presenting a specific and well distinguishable DNA fingerprinting, can be used as starting material for the tracer of the invention, to be applied on products obtained in different production periods or in different districts or by different producers, etc. E.g., variety A may be used to “label” the productions of district X in a certain year or month, and variety B may be used to “label” the products of district Y in the same year or month. The tracer of the invention can be applied externally in contact to the product to be traced at the moment of the branding, e.g. with methods approved by the producer's association and in the same area of the traditional seal. The tracer of the invention may also be applied on the wrapping of the food, on the package, on the container, on the tag containing a seal, on the seal, etc.

As easily understandable from the present description, in order to recognise the authenticity of a product sold with a certain mark or produced in a determined period from a district, it will be sufficient to verify the exact overlapping of the DNA fingerprinting of a sample of material comprising the tracer, with the one of the variety/ies used in the preparation of the tracer of the invention for that particular traceability system using one or more of the above mentioned amplification techniques and to confront the panel of alleles obtained with the expected one. If the DNA fingerprinting corresponds to the one expected for the tracer of the invention, than it will be certain that the product checked is authentic. Vice versa, the product is a fraud.

A simple analysis of the DNA of the tracer can hence allow the confirmation of the authenticity of a product.

As previously indicated, it is also object of the invention the method for the preparation of the tracer described above. In the method of preparation of the tracer, it will be possible to use various organism particularly genetically stable: it will be hence particularly advantageous to use varieties belonging to species with agamic or autogamous propagation having a high inter-variety variability.

For the process of the invention rice, olive tree or wheat varieties can be advantageously used.

In the selection of polymorphisms according to the method of the invention, it will be advantageous to use polymorphisms already known in the art, suitable for detecting without doubt the selected variety. In eukaryotes, chloroplast or mitochondrial DNA polymorphisms could be particularly advantageous.

In an embodiment of the invention, the polymorphisms can further be selected depending on their length in nucleotides wherein said length is comprised between 50 and 450 nucleotides as the amplification an the analysis of sequences comprised within said length are more easily to amplify and the amplified DNA is more easily analyzable with respect to longer sequences. For the aim of the invention sequences between 100 and 300 nucleotides are particularly advantageous.

According to the invention, the microsatellites sequences of point d. of the process for the preparation of the tracer of the invention, may be highlighted through DNA amplification with primers having an annealing temperature where the difference between the optimal annealing temperature of the primer having the lowest annealing temperature and the one of the primer having the highest annealing temperature is comprised between 0.5 e 3° C.

In an embodiment of the invention, e.g. as indicated above for the multiplex-amplifications, said temperature would be advantageously equal or higher than about 60° C. In fact, the highest is the stringency of the reactions, the lowest will be the possibility of aspecific amplification products.

In the selection of the organisms containing the polymorphic sequences, it will be advantageous, in the process of the invention, to analyze the method of manufacture of the food product to be labelled and, where suitable, to select organisms used in the production of said food product. E.g. wheat or rice flours to be admixed with pork fat used in the greasing during the preparation of hams, etc.

The tracer of the invention, comprising the natural polymorphic sequences comprising selected SNPs and/or microsatellites according to the above indicated process, may be used for the identification and the traceability of the origin of food along the whole production chain through the mere amplification of the polymorphic selected sequences comprised in the tracer of the invention by using specific primers.

According to the invention, the amplification used for the detection of the tracer and for the anti-counterfeit control or for the tracing along the whole production chain, can be effected on the tracer itself or on DNA extracted from the tracer by standard extraction techniques, using techniques such as Polymerase Chain Reaction (PCR), Real Time Polymerase Chain Reaction (RT-PCR), Isothermal and Chimeric prime-initiated Amplification of Nucleic Acids (ICAN), Rolling Circle Amplification (RCA), Invaider Technology, Single Primer Isothermal Amplification Technology (SPIA Technology), Loop Mediated Isothermal Amplification (LAMP), Ramification Amplification (RAM) and Pressure Cycling Technology (PCT). When possible, and in any case in a particularly advantageous embodiment, the detection of the tracer will be performed with a single multiplex amplification allowing the amplification of all the polymorphic sequences representative of a particular tracer of the invention in a single solution. In such a case, the primers of the invention will have similar annealing temperatures and can be labelled differentially, e.g. certain forward primers could be appropriately labelled with Fam and others with Hex, Vic and Pet.

After amplification of the target regions, various methods can be used in order to identify the alleles, e.g. with protocols based on primer extension, microsphere analysis, acrylamide gels, or others, and with different analysis platforms such as automatic sequencers, microarrays, mass spectrometers, flow cytometers etc.

The foods labelled with the tracer of the invention can be foods of any kind, from solid to liquid foods and can be labelled, as indicated above, through a labelling of material applied to the product, or through a labelling on the external surface of the product itself, or through a labelling inside the product of interest that could be, e.g., a typical product R.D.O., P.D.O., P.G.I., G.R.D.O. such as, e.g., typical hams, typical cheeses, oils, wines, various kinds of cold meats and salami, and particularly esteemed foods for which avoiding counterfeit is highly desirable.

The following examples are given to exemplify but absolutely not to limit the invention.

### EXAMPLES

### 1. Selection of the Organism to be Used for the Preparation of the Food Tracer to be Used on a Typical Product

The phases of the production cycle of dry-cured ham has been previously analyzed in order to identify which form of tracer would be the more suitable and which technological product could be used.

In the case of dry-cured ham, the tracer has been admixed with the pork fat during the greasing phase or admixed to the edible ink used for the marking.

As polymorphic DNA sources suitable for the preparation of the tracer different plant species having the following characteristics have been analyzed:
a good preservation;
a lower interference with the peculiarities of the typical product (i.e. dry-cured ham);
agamic propagating species, in order to guarantee the absence of genetic variability inside the tracer obtained from a single variety or;
autogamous propagating species having a lowest possibility of contamination from extraneous pollen;
a high inter-variety variability, so to easily recognize one variety form the other by DNA analysis;
a plant species on which molecular genetics studies had already been performed and on which a vast scientific literature already existed.

A careful bibliographical search has been effected for the selection of the organism having the sequences to be contained in the tracer and the following species have been selected: rice, wheat, and olive tree as all the three species met the desired characteristics i.e. they are: easily preservable, autogamous species (rice and wheat), or agamic (olive tree) and have a high inter-variety variability.

### 2. Selection of the Species to Use for the Preparation of the Tracer of the Invention to Label Typical Dry-Cured Ham

Taking into account that already at the stage of preparation of the pork fat for greasing wheat or rice flour is used, and that the choice can hence reasonably fall on one of the said plant species, wheat, indifferently soft or hard, has been selected in the present example. This is so, because wheat is one of the best studied species in the art for microsatellites and for which there exist a database to make reference to.

A seed industry has hence been contacted and said industry has provided about twenty soft or hard wheat varieties in pure lines, deriving from selections that did not have a commercial outcome, or from antique varieties (roman age) or from new selections, having highly preserved characters.

For each variety about twenty seeds have been allowed to sprout and DNA has been extracted from the leaves of every single plant. A homogeneity test of the DNA has been carried out, with the RAPD technique, in order to assess the pureness of each variety.

Once the homogeneity of the varieties at hand had been assessed, and once the ones with the highest homogeneity had been selected, the literature concerning SSRs for the selected species has been looked up and ten of them have been selected for testing.

### 3. Microsatellites Selection

The selection of the microsatellites has been carried out taking into account the following characteristics of SSR microsatellites:
the microsatellites had to display at least 5 alleles/SSR
the diversity index had to be higher than 0.5 taking into account the number of alleles at each polymorphic locus and the relative frequency of each allele according to the formula:

DI=1−Σpᵢ²

Where pᵢ is the frequency of the i^{th} allele.
the microsatellites had to be reproducible.
the microsatellites had to be of a length comprised between 100-300 bp so to result easily analyzable also in transformed matrix.

For the multiplex analysis, 5 microsatellites have been selected with a length between 100 and 200 bp and other 5 with a length between 200 and 300 bp.
the primers had to have similar annealing temperatures in order to carry out possible multiplex amplifications around 60° C.

### 4. Evaluation and Control of the Suitability of the Selected Microsatellites

For each harvested variety flours from 100 g of seeds have been produced and from said flours the DNA has been extracted in double and the DNA amplification of the selected SSRs has been carried out by PCR using forward and reverse primers specific for each SSR. The forward primer has been labelled with a fluorophore (6-FAM) in order to carry out the following fragments analysis. The amplification has been carried out in a final volume of 25 μl or 10 μl in a thermocycler MJ PTC-100. The master mix contained 2 picomoles of each primer, 0.2 mM of each deoxynucleotide, 1.5 Mm of MgCl₂, 1 unit of Taq Polymerase, and 50-100 ng of DNA.

The DNA has been amplified according of the following profile: 3 min at 94° C., hence 1 min at 94° C., 1 min at 60° C., 2 min at 72° C. for 35 cycles, with a final extension of 10 min at 72° C.

Subsequently, 1 μl of amplified DNA has been added to 10 μl of a formamide and ladder ROX 500 (ABI) mix and has been denatured at 95° C. for 2 min and cooled down to 4° C. after.

The fragments analysis has been carried out with an automatic sequencer at capillary electrophoresis such as, e.g. ABI 310. The polymer POP 4 (ABI) has been used for the analysis, and, given the fragments length, comprised between 100 and 300 bp, capillary of 36 cm of length were used in order to obtain a good separation of the fragments.

The DNA profiles obtained with the 10 microsatellites used have been analyzed for each variety obtaining hence a sort of genetic identity card allowing a specific characterization of each variety and allowing also to verify that said varieties were not contaminated. The varieties that did not result as pure lines have been discarded.

A database on the selected varieties has so been constructed.

### 5. Selection of the Material for the Preparation of the Tracer

Once the variety has been selected, the technological preparation that was more suitable for the application on typical dry-cured hams has been selected as well.

It has hence been decided to use, for the preparation of the tracer, a mixture of pork fat and flour of wheat of the selected variety. In vitro tests have hence been carried out on mixtures at different concentrations 1°/∞, 1%, 5% 10% in order to verify the effectiveness of the DNA extraction and amplification and the microsatellites profiles. Moreover, said tests have been carried out at different time moments, in order to verify that the effectiveness of the DNA extraction and amplification in the tracer were preserved in time and that the tracer or the DNA did not denaturate in a way that rendered the microsatellites undetectable.

### 6. Application of the Tracer on Dry-Cured Ham

Once determined the optimal conditions for detecting the tracer, the technological preparation has been directly applied onto the ham. Controls at different times have hence been carried out, by collecting tracer samples by scratching the external part of the ham. The tracer has been subjected to DNA extraction and to the subsequent microsatellites analysis. The obtained profile has been confronted with the one registered in the database and the overlapping of the profiles has confirmed the authenticity of the product and the effectiveness of the tracer of the invention.

1-18. (canceled)

19. A method of labeling a food product with a tracer comprising: labeling the food product with a tracer comprising sequences of natural origin and/or natural material and suitable excipients, wherein said DNA sequences are exogenous with respect to the food product to be labeled, and said DNA sequences comprise one or more polymorphic sequences detectable by DNA amplification at an annealing temperature equal to or higher than 50° C., said polymorphic sequences comprising SNPs and/or microsatellites, said SNPs and/or microsatellites disclose at least five alleles at each locus, and wherein a diversity index (DI) between the alleles of said loci must be equal to or higher than 0.5 taking into account the number of alleles for each microsatellite and the relative frequency of each allele according to the following formula
DI=1−Σpᵢ²
where pᵢ is the frequency of the i^{th} allele.

20. The method according to claim 19, wherein the DNA amplification is carried out by one or more techniques selected from the group consisting of Polymerase Chain Reaction (PCR), Real Time Polymerase Chain Reaction (RT-PCR), Isothermal and Chimeric prime-initiated Amplification of Nucleic Acids (ICAN), Rolling Circle Amplification (RCA), Invaider Technology, Single Primer Isothermal Amplification Technology (SPIA Technology), Loop Mediated Isothermal Amplification (LAMP), Ramification Amplification (RAM) and Pressure Cycling Technology (PCT).

21. The method according to claim 19, wherein said DNA sequences originate from a plant, animal, bacterium, fungus, yeast or other microorganism.

22. The method according to claim 19, wherein said DNA sequences derive from a species having agamic or autogamous propagation and having high inter-variety variability.

23. The method according to claim 22, wherein said species is rice, olive or wheat.

24. The method according to claim 19, wherein said microsatellite sequences are of a length comprised between 50 and 540 nucleotides.

25. The method according to claim 24, wherein said microsatellites sequences are of a length comprised between 100 and 300 nucleotides.

26. The method according to according to claim 19, wherein said polymorphic sequences may be detected by DNA amplification with primers having an annealing temperature where the difference between the optimal annealing temperature of the primer having the highest annealing temperature and the one of the primer having the lowest annealing temperature is comprised between about 0.5° C. and 3° C.

27. The method according to claim 19, wherein said annealing temperature is equal to or higher than about 60° C.

28. The method according to claim 19, in form of a solid, solution, powder, flour, lyophilized, raw plant material dry or liquid.

29. The method according to claim 19, wherein said tracer is natural material which is admixed with other food products or edible inks prior to labeling the food products.

30. A method for manufacturing a tracer for foods comprising DNA sequences of natural origin and/or natural material and suitable excipients comprising:
(a) selecting a plant variety and/or an animal line or breed and/or a fungal or bacterial or yeast or other microorganism line or strain or species comprising DNA sequences that are exogenous with respect to the DNA of the food to be traced;
(b) extracting the DNA from more than one specimen of said variety, line, breed, strain or species selected in (a), carrying out a homogeneity test for each specimen, and selecting the variety, line, breed, strain and/or species showing homogeneity;
(c) selecting one or more polymorphisms for said variety, line, breed, strain and/or species selected in (b) according to the following parameters:
said polymorphisms must comprise SNPs and/or microsatellites and said SNPs and/or microsatellites must present at least 5 alleles for each locus and
a diversity index (DI) between said loci must be equal to or higher than 0.5 taking into account the number of alleles for each locus and the relative frequency of each allele according to the following formula
DI=1−Σpᵢ²
where pᵢ is the frequency of the i^{th} allele;
(d) selecting one or more SNP and/or microsatellites selected in (c) whose DNA amplification requires annealing temperatures equal to or higher than 50° C.;
(e) isolating materials containing DNA of said variety/ies, line/s, breed/s, strain/s and/or species containing the SNPs and/or the microsatellites selected at (d) and admixing said materials with suitable excipients.

31. The method according to claim 30, wherein the DNA amplification is carried out by one or more techniques selected from the group consisting of Polymerase Chain Reaction (PCR), Real Time Polymerase Chain Reaction (RT-PCR), Isothermal and Chimeric prime-initiated Amplification of Nucleic Acids (ICAN), Rolling Circle Amplification (RCA), Invaider Technology, Single Primer Isothermal Amplification Technology (SPIA Technology), Loop Mediated Isothermal Amplification (LAMP), Ramification Amplification (RAM) and Pressure Cycling Technology (PCT).

32. The method according to claim 30, wherein said variety, line, breed, strain or species selected in (a) belongs to an agamic or a autogamous propagating species and have high inter-variety variability.

33. The method according to claim 32, wherein said variety, line, breed, strain or species selected in (a) is rice, olive or wheat.

34. The method according to claim 30, wherein said microsatellites are further selected according to their length in nucleotides and wherein said length is comprised between 50 and 450 nucleotides.

35. The method according to claim 30, wherein said polymorphic sequences can be detected by DNA amplification with primers having an annealing temperature where the difference between the optimal annealing temperature of the primer having the highest annealing temperature and the one of the primer having the lowest annealing temperature is comprised between about 0.5° C. and 3° C.

36. The method according to claim 30, wherein said annealing temperature is equal to or higher than about 60° C.

37-45. (canceled)

46. A method of identifying a food product labeled with a tracer, wherein the tracer comprises DNA sequences of natural origin and/or natural material and suitable excipients, wherein said DNA sequences are exogenous with respect to the food product to be labeled, and said DNA sequences comprise one or more polymorphic sequences detectable by DNA amplification at an annealing temperature equal to or higher than 50° C., said polymorphic sequences comprising SNPs and/or microsatellites, said SNPs and/or microsatellites disclose at least five alleles at each locus, and wherein a diversity index (DI) between the alleles of said loci must be equal to or higher than 0.5 taking into account the number of alleles for each microsatellite and the relative frequency of each allele according to the following formula
DI=1−Σpᵢ²
where pᵢ is the frequency of the i^{th} allele, comprising:
(a) amplifying polymorphic sequences of the tracer, which labels the food product;
(b) detecting the tracer from the amplified polymorphic sequences; and
(c) identifying the food product by the allele of the tracer's polymorphic sequence.
